(19) United States
(12) Patent Application Publication
(10) Pub. No.: US 2021/0259969 A1
(43) Pub. Date: Aug. 26, 2021

(54) COMPOSITIONS AND METHODS INVOLVING TRANSFORMING EXTRACELLULAR VESICLES

(71) Applicants: Michael Sabbah, Rochester, MN (US); Atta Behfar, Rochester, MN (US); Christopher Livia, Rochester, MN (US); Timothy Peterson, Rochester, MN (US); MAYO FOUNDATION FOR MEDICAL EDUCATION AND RESEARCH, Rochester, MN (US)

(72) Inventors: Michael Sabbah, Rochester, MN (US); Atta Behfar, Rochester, MN (US); Christopher Livia, Rochester, MN (US); Timothy Peterson, Rochester, MN (US)

(21) Appl. No.: 17/261,790

(22) PCT Filed: Jul. 24, 2019
(86) PCT No.: PCT/US2019/043172

§ 371 Date: Jan. 20, 2021

Related U.S. Application Data
(60) Provisional application No. 62/702,882, filed on Jul. 24, 2018.

(51) Int. Cl. A61K 9/127; A61K 31/713

(52) U.S. Cl. CPC A61K 9/1271; A61K 31/713; A61K 9/1278

(57) ABSTRACT

An extracellular vesicle includes an exogenous therapeutic component. The exogenous therapeutic component can include a therapeutic polypeptide, a polynucleotide that encodes a therapeutic polypeptide, a therapeutic nucleic acid, or a therapeutic agent. In some embodiments, the extracellular vesicle includes an exosome or purified exosome product (PEP).

### CROSS-REFERENCE TO RELATED APPLICATION

This application claims the benefit of U.S. Provisional Patent Application No. 62/702,882, filed Jul. 24, 2018, which is incorporated herein by reference in its entirety.

### SUMMARY

This disclosure describes, in one aspect, an extracellular vesicle that includes an exogenous therapeutic component. The exogenous therapeutic component can include a therapeutic polypeptide, a polynucleotide that encodes a therapeutic polypeptide, a therapeutic nucleic acid, or a therapeutic agent.

In some embodiments, the extracellular vesicle includes an exosome or purified exosome product (PEP).

In some embodiments, the therapeutic nucleic acid includes a native RNA, a native DNA, plasmid DNA, modified plasmid DNA, modified miRNA, modified mRNA, modified DNA, an inhibitory RNA, a small interfering RNA (siRNA), a short hairpin RNA (shRNA), a Y RNA, a long non-coding RNA (lncRNA), an agomiR, or an antagomiR.

In some embodiments, the native RNA encodes a therapeutic peptide or a therapeutic protein.

In some embodiments, the therapeutic component can include a native or a heterologous protein.

In another aspect, this disclosure describes a method of transforming an extracellular vesicle. Generally, the method includes obtaining extracellular vesicles, providing a therapeutic agent of interest, and introducing the therapeutic agent of interest into at least a portion of the extracellular vesicles.

In some embodiments, the extracellular vesicle includes an exosome or purified exosome product (PEP).

In some embodiments, the therapeutic agent of interest is introduced into the extracellular vesicle by electroporation.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1. Overview of exemplary processes in the synthesis of exosomes/extracellular vesicles being enhanced by transformation with heterologous materials.

FIG. 2. “DNA” column represents luminescence of mouse skeletal muscle injected with modified-DNA that was mixed with a “traditional” transfection reagent (positive control, no electroporation). “PEP DNA” column represents luminescence of mouse skeletal muscle injected with modified DNA that underwent electroporation transfection. RLU=Relative Light Units.

FIG. 3. PEP labeled with a non-specific lipid dye (red, Thermo-Fischer Scientific) can be seen within the cell membrane in vitro.

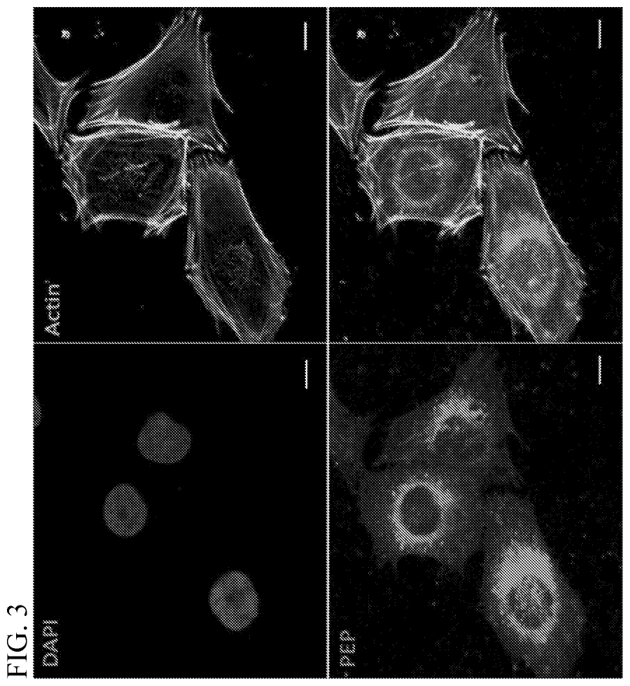

FIG. 4. Solvent dispersion and lipid film preparation of PEP followed by hydration of the lipids with PBS and DNA encoding GFP. Freeze-dried lipid film was rehydrated overnight and added to low-serum media of 293T cells, demonstrating delivery of GFP encoding DNA plasmid.

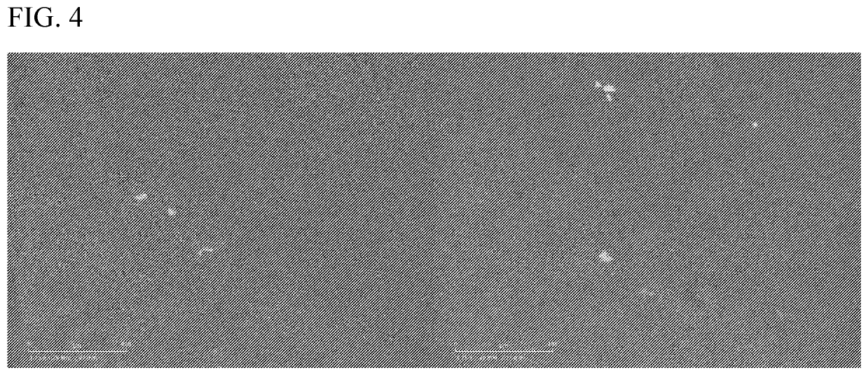

### DETAILED DESCRIPTION OF ILLUSTRATIVE EMBODIMENTS

Exosomes and other extracellular vesicles have powerful angiogenic and restorative properties. This disclosure describes methods by which these vesicles can be further enhanced by adding DNA, RNA, protein, or a therapeutic agent to the vesicle. For example, PEP (Purified Exosome Product, PCT/US2018/065627; WO 2019/118817 A1) or an extracellular vesicle can be further enhanced with RNA to give it anti-inflammatory properties using a process sometimes referred to as transfection.

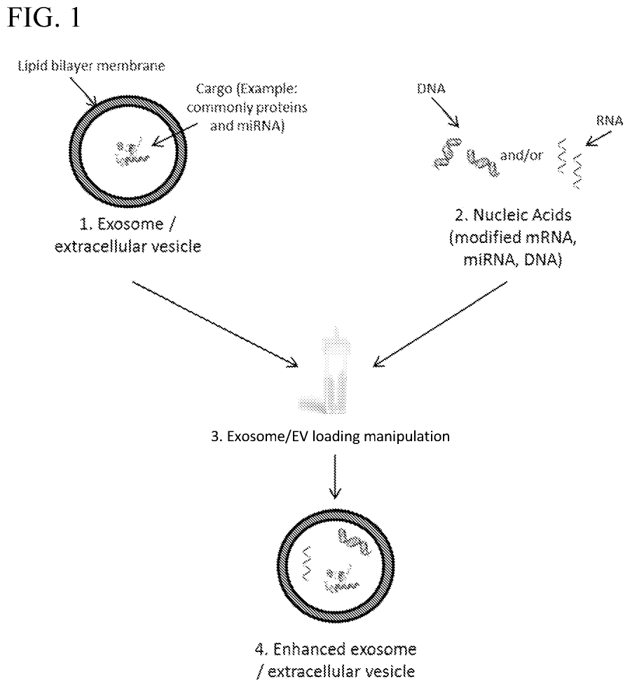

An exemplary process for transfecting an extracellular vesicle is shown schematically in FIG. 1. An extracellular vesicle is shown as a lipid bilayer membrane enveloping cargo. The cargo, as illustrated in FIG. 1, can include miRNA, DNA, known drugs, and/or protein derivatives.

The transfection process introduces a nucleic acid, protein, or agent of interest into the extracellular vesicle. A nucleic acid of interest may be any suitable DNA, RNA, or a mixture of DNA and RNA. A nucleic acid can encode, for example, a chemotherapeutic drug or protein. Alternatively, the extracellular vesicle may be transformed by introducing a purified protein or other therapeutic agent (e.g., a chemotherapeutic agent). The transformed extracellular vesicles may be used as a therapeutic agent to, for example, deliver the transforming nucleic acid to damaged tissue.

In the exemplary embodiment illustrated in FIG. 1, extracellular vesicles are transfected with nucleic acid by electroporation, result in an enhanced extracellular vesicle that includes the original cargo molecules and the nucleic acid or nucleic acids of interest.

FIG. 1 illustrates just one exemplary embodiment of a more general platform. For example, the extracellular vesicle shown in FIG. 1 can be any suitable extracellular vesicle. In certain embodiments, the extracellular vesicle being transformed may be, but is not limited to, an exosome, PEP (PCT/US2018/065627; WO 2019/118817 A1), extracellular vesicles isolated from mesenchymal stem cells, extracellular vesicles isolated from dendritic cells, extracellular vesicles isolated from cardiomyocytes, extracellular vesicles from vascular smooth muscle, extracellular vesicles isolated from endothelial cells, extracellular vesicles isolated from fibroblasts, or extracellular vesicles isolated from leukocytes.

Thus, the extracellular vesicle may be any suitable extracellular vesicle including, but not limited to, exosomes or PEP. For example, PEP has many tissue regenerative properties (PCT/US2018/065627; WO 2019/118817 A1). Moreover, PEP possesses unique biophysical properties that render PEP particularly amenable to transformation with nucleotides and/or proteins. In some embodiments, therefore, transformed PEP may be engineered to deliver additional therapeutic properties

Similarly, exosomes have been investigated as drug delivery vehicles. Exosomes may be transfected with nucleic acid that, when the exosome is taken up by a cell, effectively transforms that target cell to express the therapeutic peptide, protein, or nucleic acid encoded by the nucleic acid carried to the cell by the transformed exosome.

Exemplary nucleic acids include, but are not limited to, DNA, RNA, or modified DNA, and/or modified mRNA. Modified mRNAs are described in International Patent Application No. PCT/US2017/063060 (International Publication No. WO 2018/098312) and in International Patent Application No. PCT/US2019/033705, entitled “MICROENCAPSULATED MODIFIED POLYNUCLEOTIDE COMPOSITIONS AND METHODS,” filed May 23, 2019.

The nucleic acid, as noted above, can include any suitable form of nucleic acid including, but not limited to, a native RNA, a native DNA, a plasmid DNA, a modified plasmid DNA, a modified miRNA, a modified mRNA, a modified DNA, an inhibitory RNAs (e.g., an antisense RNA, a microRNA (miRNA), a small interfering RNA (siRNA), a short hairpin RNA (shRNA)), a Y RNA, a long non-coding RNA (lncRNA), an agomiR, or an antagomiR, or any combination thereof. As used herein, “native” RNA or “native” DNA refers to RNA or DNA that is isolated without modification. In contrast, a “modified” nucleic acid, whether RNA or DNA, can be modified to contain certain coding regions such as, for example, modified to encode for a therapeutic protein (e.g., VEGF) and/or contain one or more elements that may modify the half-life or expression level of the nucleic acid.

The nucleic acid can encode any suitable therapeutic peptide, protein, or RNA. Likewise, the protein or therapeutic agent can be any suitable therapeutic protein or other agent. For example, an extracellular vesicle can be transfected to include one or more polypeptides—or one or more mRNAs that encode a polypeptide—useful for regenerating cardiac function and/or tissue. Examples of polypeptides that can be useful for regenerating cardiac function and/or tissue include, without limitation, TNF-α, mitochondrial complex-1, resolvin-D1, NAP-2, TGF-α, ErBb3, VEGF, IGF-1, FGF-2, PDGF, IL-2, CD19, CD20, CD80/86, polypeptides described in WO 2015/034897, or an antibody directed against any of the foregoing polypeptides. For example, a human Nap-2 polypeptide can have the amino acid sequence set forth in, for example, National Center for Biotechnology Information (NCBI) Accession No. NP_002695.1 (GI No. 5473) and can be encoded by the nucleic acid sequence set forth in NCBI Accession No. NM_002704 (GI No. 5473). In some cases, a human TGF-α polypeptide can have the amino acid sequence set forth in NCBI Accession No. NP_003227.1 (GI No. 7039) and can be encoded by the nucleic acid sequence set forth in NCBI Accession No. NM_003236 (GI No. 7039). In some cases, a human ErBb3 polypeptide can have the amino acid sequence set forth in NCBI Accession No. NP_001005915.1 or NP_001973.2 (GI No. 2065) and can be encoded by the nucleic acid sequence set forth in NCBI Accession No. NM_001005915.1 or NM_001982.3 (GI No. 2065). For example, a human VEGF can have the amino acids set forth in NCBI Accession Nos. AAA35789.1 (GI: 181971), CAA44447.1 (GI: 37659), AAA36804.1 (GI: 340215), or AAK95847.1 (GI: 15422109), and can be encoded by the nucleic acid sequence set forth in NCBI Accession No. AH001553.1 (GI: 340214). For example, a human IGF-1 can have the amino acid sequence set forth in NCBI Accession No. CAA01954.1 (GI: 1247519) and can be encoded by the nucleic acid sequence set forth in NCBI Accession No. A29117.1 (GI: 1247518). For example, a human FGF-2 can have the amino acid sequence set forth in NCBI Accession No. NP_001997.5 (GI: 153285461) and can be encoded by the nucleic acid sequence set forth in NCBI Accession No. NM_002006.4 (GI: 153285460). For example, a human PDGF can have the amino acid sequence set forth in NCBI Accession No. AAA60552.1 (GI: 338209) and can be encoded by the nucleic acid sequence set forth in NCBI Accession No. AH002986.1 (GI: 338208). For example, a human IL-2 can have the amino acid sequence set forth in NCBI Accession No. AAB46883.1 (GI: 1836111) and can be encoded by the nucleic acid sequence set forth in NCBI Accession No. 577834.1 (GI: 999000). For example, a human CD19 can have the amino acid sequence set forth in NCBI Accession No. AAA69966.1 (GI: 901823) and can be encoded by the nucleic acid sequence set forth in NCBI Accession No. M84371.1 (GI: 901822). For example, a human CD20 can have the amino acid sequence set forth in NCBI Accession No. CBG76695.1 (GI: 285310157) and can be encoded by the nucleic acid sequence set forth in NCBI Accession No. AH003353.1 (GI: 1199857). For example, a human CD80 can have the amino acid sequence set forth in NCBI Accession No. NP_005182.1 (GI: 4885123) and can be encoded by the nucleic acid sequence set forth in NCBI Accession No. NM_005191.3 (GI: 113722122), and a human CD86 can have the amino acid sequence set forth in NCBI Accession No. AAB03814.1 (GI: 439839) and can be encoded by the nucleic acid sequence set forth in NCBI Accession No. CR541844.1 (GI: 49456642). For example, a polypeptide that can be useful for regenerating cardiac function and/or tissue can be an antibody directed against TNF-α, mitochondrial complex-1, or resolvin-D1.

Other suitable proteins include an antibody or a fragment thereof. An antibody, whether used to transform the extracellular vesicle or encoded by a nucleic acid used to transform the extracellular vesicle—may be a conventional full antibody, an antibody fragment, or a chimeric antibody such as, for example, a Fab, F(ab′)₂, Fab′,scFv,di-scFv, sdAb,bi-functional antibody (e.g., a BiTE or BiKE), or trifunctional antibody (e.g., TriTE or TriKE). In one illustrative embodiment, PEP may be loaded with an antibody such as rituximab to provide combination therapy.

In some cases, an extracellular vesicle can be transfected to include one or more inhibitory RNAs useful to treat a mammal experiencing a major adverse cardiac event (e.g., acute myocardial infarction) and/or a mammal at risk of experiencing a major adverse cardiac event (e.g., patients who underwent PCI for STEMI). For example, an extracellular vesicle can be transfected to include one or more inhibitory RNAs inhibiting and/or reducing expression of one or more of the following polypeptides: eotaxin-3, cathepsin-S, DK-1, follistatin, ST-2, GRO-α, IL-21, NOV, transferrin, TIMP-2, TNFαRI, TNFαRII, angiostatin, CCL25, ANGPTL4, MMP-3, and polypeptides described in WO 2015/034897. For example, a human eotaxin-3 polypeptide can have an amino acid sequence set forth in, for example, NCBI Accession No: No. NP_006063.1 (GI No. 10344) and can be encoded by the nucleic acid sequence set forth in NCBI Accession No. NM_006072 (GI No. 10344). In some cases, a human cathepsin-S can have the amino acid sequence set forth in NCBI Accession No. NP_004070.3 (GI No. 1520) and can be encoded by the nucleic acid sequence set forth in NCBI Accession No. NM_004079.4 (GI No. 1520). In some cases, a human DK-1can have the amino acid sequence set forth in NCBI Accession No. NP_036374.1 (GI No. 22943) and can be encoded by the nucleic acid sequence set forth in NCBI Accession No. NM_012242 (GI No. 22943). In some cases, a human follistatin can have then amino acid sequence set forth in NCBI Accession No. NP_037541.1 (GI No. 10468) and can be encoded by the nucleic acid sequence set forth in NCBI Accession No. NM_013409.2 (GI No. 10468). In some cases, a human ST-2 can have the amino acid sequence set forth in NCBI Accession No. BAA02233 (GI No. 6761) and can be encoded by the nucleic acid sequence set forth in NCBI Accession No D12763.1 (GI No 6761). In some cases, a human GRO-α polypeptide can have the amino acid sequence set forth in NCBI Accession No. NP_001502.1 (GI No. 2919) and can be encoded by the nucleic acid sequence set forth in NCBI Accession No. NM_001511 (GI No. 2919). In some cases, a human IL-21 can have the amino acid sequence set forth in NCBI Accession No. NP_068575.1 (GI No. 59067) and can be encoded by the nucleic acid sequence set forth in NCBI Accession No. NM_021803 (GI No. 59067). In some cases, a human NOV polypeptide can have the amino acid sequence set forth in NCBI Accession No. NP_002505.1 (GI No. 4856) and can be encoded by the nucleic acid sequence set forth in NCBI Accession No. NM_002514 (GI No. 4856). In some cases, a human transferrin polypeptide can have the amino acid sequence set forth in NCBI Accession No. NP_001054.1 (GI No. 7018) and can be encoded by the nucleic acid sequence set forth in NCBI Accession No. NM_001063.3 (GI No. 7018). In some cases, a human TIMP-2 polypeptide can have the amino acid sequence set forth in NCBI Accession No. NP_003246.1 (GI No. 7077) and can be encoded by the nucleic acid sequence set forth in NCBI Accession No. NM_003255.4 (GI No. 7077). In some cases, a human TNFαRI polypeptide can have the amino acid sequence set forth in NCBI Accession No. NP_001056.1 (GI No. 7132) and can be encoded by the nucleic acid sequence set forth in NCBI Accession No. NM_001065 (GI No. 7132). In some cases, a human TNFαRII polypeptide can have the amino acid sequence set forth in NCBI Accession No. NP_001057.1 (GI No. 7133) and can be encoded by the nucleic acid sequence set forth in NCBI Accession No. NM_001066 (GI No. 7133). In some cases, a human angiostatin polypeptide can have the amino acid sequence set forth in NCBI Accession No. NP_000292 (GI No. 5340) and can be encoded by the nucleic acid sequence set forth in NCBI Accession No. NM_000301 (GI No. 5340). In some cases, a human CCL25 polypeptide can have the amino acid sequence set forth in NCBI Accession No. NP_005615.2 (GI No. 6370) and can be encoded by the nucleic acid sequence set forth in NCBI Accession No. NM_005624 (GI No. 6370). In some cases, a human ANGPTL4 polypeptide can have the amino acid sequence set forth in NCBI Accession No. NP_001034756.1 or NP_647475.1 (GI No. 51129) and can be encoded by the nucleic acid sequence set forth in NCBI Accession No. NM_001039667.1 or NM_139314.1 (GI No. 51129). In some cases, a human MMP-3 polypeptide can have the amino acid sequence set forth in NCBI Accession No. NP_002413.1 (GI No. 4314) and can be encoded by the nucleic acid sequence set forth in NCBI Accession No. NM_002422 (GI No. 4314).

In some cases, an extracellular vesicle can be transfected to include one or more nucleotides that modulate (e.g., mimic or inhibit) microRNAs involved in cardiac regenerative potency. For example, an extracellular vesicle can be transfected to include one or more agomiRs that mimic one or more miRNAs to augment cardiac regenerative potency. For example, an extracellular vesicle can be transfected to include one or more antagomiRs that inhibit one or more miRNAs to augment cardiac regenerative potency. Examples of miRNAs involved in cardiac regenerative potency include, without limitation, miR-127, miR-708, miR-22-3p, miR-411, miR-27a, miR-29a, miR-148a, miR-199a, miR-143, miR-21, miR-23a-5p, miR-23a, miR-146b-5p, miR-146b, miR-146b-3p, miR-2682-3p, miR-2682, miR-4443, miR-4443, miR-4521, miR-4521, miR-2682-5p,miR-2682, miR-137.miR-137, miR-549.miR-549, miR-335-3p, miR-335, miR-181c-5p, miR-181c, miR-224-5p, miR-224, miR-3928, miR-3928, miR-324-5p, miR-324, miR-548h-5p, miR-548h-1, miR-548h-5p, miR-548h-2, miR-548h-5p, miR-548h-3, miR-548h-5p, miR-548h-4, miR-548h-5p, miR-548h-5, miR-4725-3p, miR-4725, miR-92a-3p, miR-92a-1, miR-92a-3p, miR-92a-2, miR-134, miR-134, miR-432-5p, miR-432, miR-651, miR-651, miR-181a-5p, miR-181a-1, miR-181a-5p, miR-181a-2, miR-27a-5p, miR-27a, miR-3940-3p, miR-3940, miR-3129-3p, miR-3129, miR-146b-3p, miR-146b, miR-940, miR-940, miR-484, miR-484, miR-193b-3p, miR-193b, miR-651, miR-651, miR-15b-3p, miR-15b, miR-576-5p, miR-576, miR-377-5p, miR-377, miR-1306-5p, miR-1306, miR-138-5p, miR-138-1, miR-337-5p, miR-337, miR-135b-5p, miR-135b, miR-16-2-3p, miR-16-2, miR-376c.miR-376c, miR-136-5p, miR-136, let-7b-5p, let-7b, miR-377-3p, miR-377, miR-1273g-3p, miR-1273g, miR-34c-3p, miR-34c, miR-485-5p, miR-485, miR-370.miR-370, let-7f-1-3p, let-7f-1, miR-3679-5p, miR-3679, miR-20a-5p, miR-20a, miR-585.miR-585, miR-3934, miR-3934, miR-127-3p, miR-127, miR-424-3p, miR-424, miR-24-2-5p, miR-24-2, miR-130b-5p, miR-130b, miR-138-5p, miR-138-2, miR-769-3p, miR-769, miR-1306-3p, miR-1306, miR-625-3p, miR-625, miR-193a-3p, miR-193a, miR-664-5p, miR-664, miR-5096.miR-5096, let-7a-3p, let-7a-1, let-7a-3p, let-7a-3, miR-15b-5p, miR-15b, miR-18a-5p, miR-18a, let-7e-3p, let-7e, miR-1287.miR-1287, miR-181c-3p, miR-181c, miR-3653, miR-3653, miR-15b-5p, miR-15b, miR-1, miR-1-1, miR-106a-5p, miR-106a, miR-3909.miR-3909, miR-1294.miR-1294, miR-1278, miR-1278, miR-629-3p, miR-629, miR-340-3p, miR-340, miR-200c-3p, miR-200c, miR-22-3p, miR-22, miR-128, miR-128-2, miR-382-5p, miR-382, miR-671-5p, miR-671, miR-27b-5p, miR-27b, miR-335-5p, miR-335, miR-26a-2-3p, miR-26a-2, miR-376b.miR-376b, miR-378a-5p, miR-378a, miR-1255a, miR-1255a, miR-491-5p, miR-491, miR-590-3p, miR-590, miR-32-3p, miR-32, miR-766-3p, miR-766, miR-30c-2-3p, miR-30c-2, miR-128.miR-128-1, miR-365b-5p, miR-365b, miR-132-5p, miR-132, miR-151b.miR-151b, miR-654-5p, miR-654, miR-374b-5p, miR-374b, miR-376a-3p, miR-376a-1, miR-376a-3p, miR-376a-2, miR-149-5p, miR-149, miR-4792.miR-4792, miR-1.miR-1-2, miR-195-3p, miR-195, miR-23b-3p, miR-23b, miR-127-5p, miR-127, miR-574-5p, miR-574, miR-454-3p, miR-454, miR-146a-5p, miR-146a, miR-7-1-3p, miR-7-1, miR-326.miR-326, miR-301a-5p, miR-301a, miR-3173-5p, miR-3173, miR-450a-5p, miR-450a-1, miR-7-5p, miR-7-1, miR-7-5p, miR-7-3, miR-450a-5p, miR-450a-2, miR-1291, miR-1291, miR-7-5p, miR-7-2, and miR-17-5p, or miR-17.

Nucleotides (e.g., RNA) used to transfect an extracellular vesicle can be modified nucleotides. In some cases, nucleotides can be modified for increased stability. For example, one or more uracil residues of an RNA described herein can be replace with a modified uracil residue. Examples of modified uracil residues include, without limitation, pseudouridine (T), dihydrouridine (D), and dideoxyuracil. An mRNA may be modified to form a biofunctionalized microencapsulated modified mRNA (M³RNA), which are described in more detail in International Patent Application No. PCT/US2017/063060, filed Nov. 22, 2017, entitled “PARTICLE-MEDIATED DELIVERY OF BIOLOGICS,” which published as International Publication No. WO 2018/098312, and in in International Patent Application No. PCT/US2019/033705, entitled “MICROENCAPSULATED MODIFIED POLYNUCLEOTIDE COMPOSITIONS AND METHODS,” filed May 23, 2019.

The therapeutics listed above are merely exemplary. Other therapeutics, including miRNAs, can includes therapeutic agents that target organs outside of the cardiovascular system. The nucleic acid can be introduced into the extracellular vesicle by any suitable method. FIG. 1 illustrates an exemplary embodiment in which nucleic acids are introduced into the extracellular vesicle by electroporation. Alternative suitable methods for introducing nucleic acid into the extracellular vesicle include active loading techniques or passive loading techniques. Exemplary active loading techniques include, for example, electroporation, chemical-gradient coupled loading, osmotic-gradient coupled loading, or pH-dependent loading. Exemplary passive loading techniques include, but are not limited to, a mechanical dispersion method (e.g., lipid film hydration, micro emulsification, sonication, French pressure cell, membrane extrusion, dried reconstituted vesicles, freeze-thawed liposomes), a solvent dispersion method (e.g., microfluidic loading, ethanol injection, ether injection, double emulsion, reverse phase evaporation, stable pluri lamellar vesicles), a detergent removal methods (using, e.g., cholate, alkylglycoside, triton X-100), or removal from mixed vesicles (e.g., dialysis, column chromatography, dilution, reconstituted sendai virus envelope).

After the nucleic acid is introduced into the extracellular vesicle, the transformed product may be stored for future use. For example, one can freeze dry or lyophilize the transformed product to increase the shelf-life of the transformed product.

The transformed extracellular vesicles may be used as a therapeutic agent to, for example, deliver the transforming nucleic acid to damaged tissue. For example, PEP has many tissue regenerative properties (PCT/US2018/065627; WO 2019/118817 A1). Transformed PEP may be engineered to deliver additional therapeutic properties. Moreover, PEP possesses unique biophysical properties that render PEP particularly amenable to transformation with nucleotides and/or proteins.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

Purified exosomes (PEP) were prepared as previously described (International Patent Application Publication PCT/US2018/065627, published as WO 2019/118817 A1). The PEP was reconstituted using sterile water and heparin sulfate (1000 units/mL), and then filtered through a 0.2 μm filter. 20 μl of reconstituted and filtered PEP was added to a 1-mm electroporation cuvette. 12 μg of mod-DNA encoding for a reporter gene was obtained from GenScript (Piscataway, N.J.) and added to electroporation cuvette. The reporter gene encodes nano-luciferase, which generates a light signal in the presence of enzyme substrate, furimazine (Promage, Madison, Wis.)

The cuvette was placed in an electroporator (GENEPULSER XCELL, Bio-Rad Laboratories, Inc., Hercules, Calif.), and electroporation was performed with the following settings: 350v, 150 uF, 1 pulse. The electroporation product was removed from the cuvette, pipetted into an Eppendorf tube, and placed on ice for 10 minutes.

The final product was injected into the thigh muscle of a mouse. 48 hours after the product was injected, muscle from the injection site was collected. Muscle from a distant site was collected as a control. The injected muscle tissue and control muscle tissue were then processed as instructed in the NANO-GLO luciferase kit (Promega, Madison, Wis.), and analyzed via plate reader (FLUOSTAR OMEGA, BMG Labtech) following manufacturer's instructions for downstream protein content via plate reader.

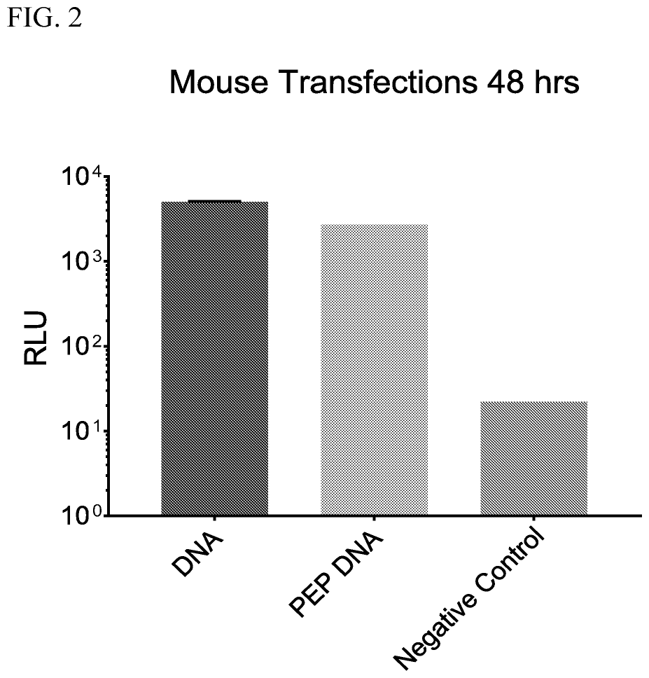

Results are shown in FIG. 2.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. An extracellular vesicle comprising an exogenous therapeutic component, the exogenous therapeutic component comprising:
a therapeutic polypeptide;
a polynucleotide that encodes a therapeutic polypeptide;
a therapeutic nucleic acid; or
a therapeutic agent.

2. The extracellular vesicle of claim 1, wherein the extracellular vesicle comprises an exosome or purified exosome product (PEP).

3. The extracellular vesicle of claim 1, wherein the therapeutic nucleic acid comprises native RNA, native DNA, plasmid DNA, modified plasmid DNA, modified miRNA, modified mRNA, modified DNA, an inhibitory RNA, a small interfering RNA, a short hairpin RNA, a Y RNA, a long non-coding RNA, an agomiR, or an antagomiR.

4. The extracellular vesicle of claim 3, wherein the native DNA or the native RNA encodes a therapeutic peptide or a therapeutic protein.

5. A method of transforming an extracellular vesicle, the method comprising:
obtaining extracellular vesicles;
providing a therapeutic agent of interest; and
introducing the therapeutic agent of interest into at least a portion of the extracellular vesicles.

6. The method of claim 5, wherein the polynucleotide of interest comprises native RNA, native DNA, plasmid DNA, modified plasmid DNA, modified miRNA, modified mRNA, modified DNA, an inhibitory RNA, a small interfering RNA, a short hairpin RNA, an agomiR, or an antagomiR.

7. The method of claim 6, wherein the native DNA or the native RNA encodes a therapeutic peptide or a therapeutic protein.

8. The method of claim 6, wherein the therapeutic agent comprises a therapeutic protein.

9. The method of claim 5, wherein the extracellular vesicles comprise an exosome or purified exosome product (PEP).

10. The method of claim 5, wherein the therapeutic agent of interest is introduced into the extracellular vesicle by electroporation.

11. A method of delivering a therapeutic agent to a cell of a subject, the method comprising:
providing a composition comprising the extracellular vesicle of claim 1; and
contacting the extracellular vesicle with a cell of the subject;
allowing the cell of the subject to take up the extracellular vesicle and release the exogenous therapeutic component into the cell.

12. The method of claim 11, wherein:
the exogenous therapeutic component comprises a therapeutic nucleic acid or a polynucleotide that encodes a therapeutic polypeptide; and
the method further includes allowing the cell to express the therapeutic polypeptide or the therapeutic nucleic acid.

13. A method of delivering a therapeutic agent to an extracellular space of a subject, the method comprising:
providing a composition comprising the extracellular vesicle of claim 1; and
contacting the extracellular vesicle with the extracellular space of the subject in need of treatment;
allowing the extracellular vesicle to occupy the extracellular space of the subject in need of treatment and release the exogenous therapeutic component into the extracellular space.
